(19) United States
(12) Patent Application Publication
(10) Pub. No.: US 2010/0145464 A1
(43) Pub. Date: Jun. 10, 2010

(54) DEVICE AND METHOD FOR FORCE MANAGEMENT WITHIN A JOINT

(76) Inventor: John-William Sidhom, Westfield, NJ (US)

Correspondence Address: SCHOX PLC, 500 3rd Street, Suite 515, San Francisco, CA, 94107, US

(21) Appl. No.: 12/329,494

(22) Filed: Dec. 5, 2008

(51) Int. Cl. A61F 2/30

(52) U.S. Cl. 623/18.12

(57) ABSTRACT

Disclosed is a device and method of management of forces within a joint. The device includes a first component with a first magnet arrangement providing a first magnetic field, a second component to interface with the first component with a second magnet arrangement providing a second magnetic field, and a compressible volume that is coupled with the second component that controls the separation of the first and second magnetic fields based upon a compressive force that causes the compressible volume to compress. The method includes using the normal force generated between the first and second components during joint use as the compressive force, causing the compressible volume to compress, bringing the first and a second magnetic fields into contact and overlap, and creating forces to couple with the normal force and regulating the overall normal force between the first and second components.

### TECHNICAL FIELD

This invention relates generally to the medical implant field, and more specifically to a new and useful device and method for force management within a joint in the field of artificial joints for medical implantation.

### BACKGROUND

In the field of skeletal joint rehabilitation, artificial joints have been used to replace damaged joints in the human body in order to alleviate pain and allow the patient to regain normal mobility that may have been lost due to joint damage. There has been much iteration in artificial joint design for various parts of the body, for example, the shoulder, the knee, the hip, etc. Despite the progress that has been seen in artificial joint design in recent history, several challenges remain that prevent artificial joints from lasting and functioning as well as a healthy natural joint, necessitating repair and/or replacement of the artificial joint.

A major challenge that currently exists in artificial joint design is wear. Artificial joints generally consist of two major components that are made of materials that aim to minimize the coefficient of friction between the two major components and mimic that of cartilage and fluid in a natural joint. The minimization of friction between the two major components of an artificial joint accomplishes two major functions: to extend the life of the joint and to minimize wear at the interface of the two major components. With wear comes the creation of wear particles. Once the wear particles become numerous, the immune system within the body functions to send macrophages to the site of the artificial join and attack the wear particles and consequently also attack healthy bone and tissue, resulting in resorption of the healthy bone and tissue and causing further bone loss and damage to the joint site. This problem perpetuates because the more the interface of the two major components is worn down, the more wear particles are generated because the surfaces between the two major components are no longer the smooth surfaces of a new artificial joint. Because artificial joints are anchored to healthy bone, as additional bone becomes reabsorbed around the artificial joint, the artificial joint starts to loosen from the implant site (potentially causing further wear). Eventually, the joint will need to be replaced. The problem of wear also exists in joints used in other applications such as machines, linkages, mechanical bearings, and braces.

Investigations into minimizing the friction, and thus the wear, between the two major components of an artificial joint have lead to innovations in new materials for the interface of the two major components. However, even with extremely low coefficients of frictions between the materials at the interface of the joint, wear particles are still produced. In addition, the new materials may have other detrimental properties such as low fracture resistance, brittleness, unknown long-term biocompatibility with the body, etc.

Thus, there is a need in the medical implant field to create a new and useful device and method for force management within a joint to minimize wear within an artificial joint while remaining biocompatible, robust, and durable. This invention provides such a new and useful device and method.

### BRIEF DESCRIPTION OF THE FIGURES

FIG. 1 is a schematic representation of the preferred embodiments of the invention;

FIG. 2 is a schematic representation of the force interactions within the preferred embodiments of the invention;

FIG. 3 is a schematic representation of the first preferred embodiment of the invention;

FIG. 4 is a schematic representation of the second preferred embodiment of the invention; and

FIG. 5 is a schematic representation of the third preferred embodiment of the invention.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

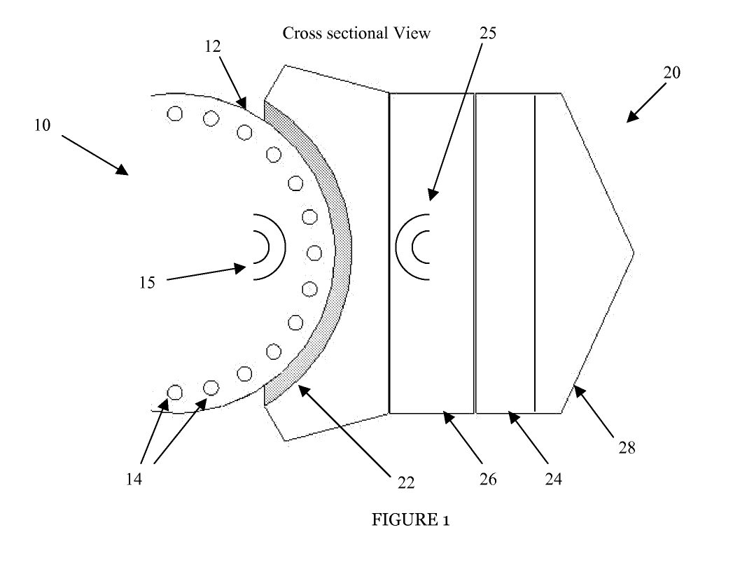

As shown in FIG. 1, the artificial joint of the preferred embodiments includes a first component 10 that includes a first component joint interface 12 and a first component magnet arrangement 14 that creates a first magnetic field 15; a second component 20 that includes a second component joint interface 22 and a second component magnet arrangement 24 that creates a second magnetic field 25; and a compressible volume 26 located between the first and second magnet arrangements 14 and 24 and adapted to control the overlap of the first and second magnetic fields 15 and 25. The first component joint interface 12, the compressible volume 26, and the second component joint interface 22 cooperate to transfer compression loads. Under a relatively low compression force, the compressible volume 26 reaches a low compression state, and the first magnetic field 15 and the second magnetic field 25 create a low repulsion force that acts upon the relatively low compression force. Under a relatively high compression force, however, the compressible volume 26 reaches a high compression state that is more compressed than the low compression state, and the first magnetic field 15 and the second magnetic field 25 create a high repulsion force that is greater than the low repulsion force and that acts upon the relatively high compression force. The high repulsive force decreases the friction force between the first and second components, thus decreasing wear in the artificial joint.

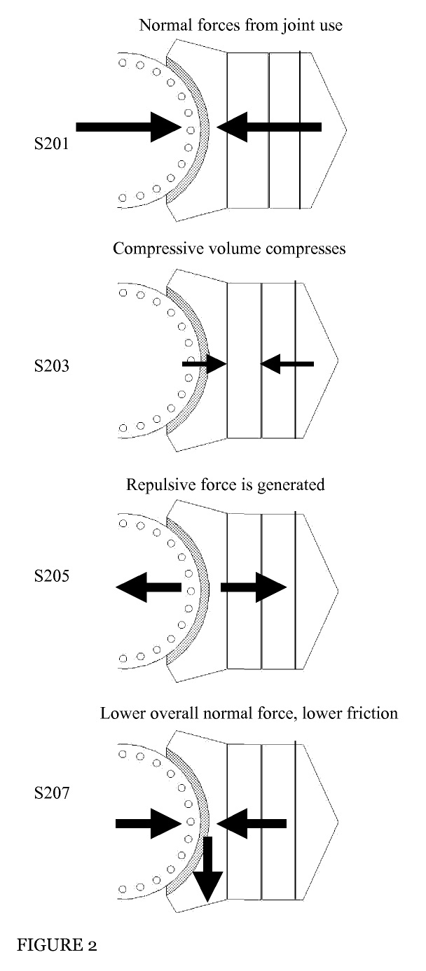

In conventional artificial joints, the coupling between the first and second joint interfaces during use create perpendicular (or “normal”) forces on the first and second components. As the first and second joint interfaces move relative to each other when there are applied normal forces, a friction force is created between the first and second joint interfaces, resulting in wear on the first and second joint interfaces. In the preferred embodiments, as shown in FIG. 2, normal force is generated on the first and second components 10 and 20 in Step S201, which result in compressive forces felt by the compressible volume 26, which cause the compressible volume 26 to compress in Step S203. The compression of the compressible volume 26 brings the first and second magnetic fields 15 and 25 closer together. The first and second magnetic fields 15 and 25 are preferably arranged such that contact and overlap of the two magnetic fields 15 and 25 creates a repulsive force in a direction opposite of the normal force created by the coupling between the first and second components 10 and 20 in Step S205, resulting in reduced overall normal force, Step S207. As a result, the overall friction force is decreased due to the decreased overall normal force, reducing wear on the first and second joint interfaces 12 and 22. Because the overall normal force is reduced, the repulsive forces may be used to extend the life and usability for materials that succumb to high normal forces. Alternatively, the first and second magnetic fields 15 and 25 may be arranged to create an attractive force upon contact and overlap in situations where the first and second joint interfaces 12 and 22 benefit from an increased normal force. For example, the first joint interface 12 may be designed to have geometry where a certain portion of the interface 12 may be better suited for higher normal forces than another. The magnetic fields 15 and 25 can then be used to direct normal forces towards this portion of the interface 12. The first and second magnetic fields 15 and 25 may also be a combination of attractive and repulsive forces along the range of the magnetic fields. For example, within the range of motion of the joint, the first and second magnetic fields 15 and 25 may be attractive within certain portions of the range and may be repulsive in other portions of the range, resulting in force management within the joint that is specific to motion type, motion range, and location of contact point between the first and second components 10 and 20. However, any other type of magnetic field 15 and 25 suitable for the application may be used.

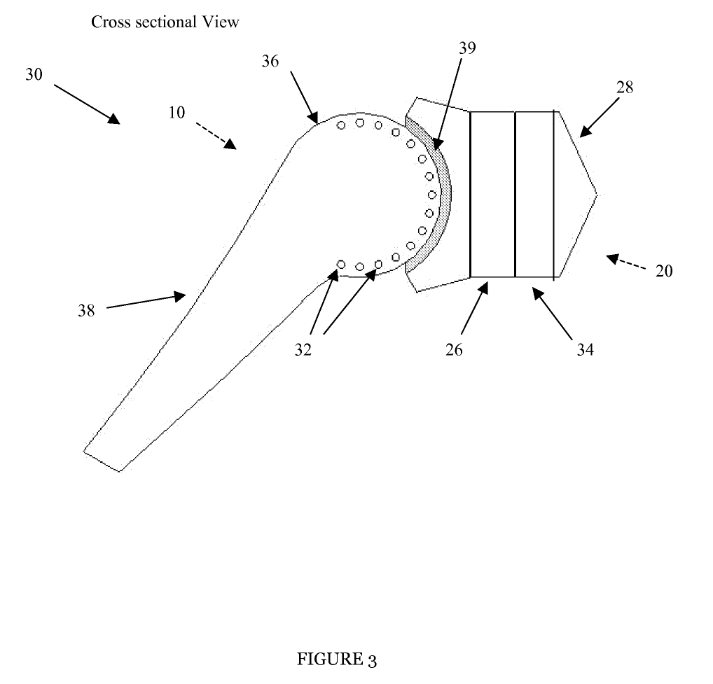

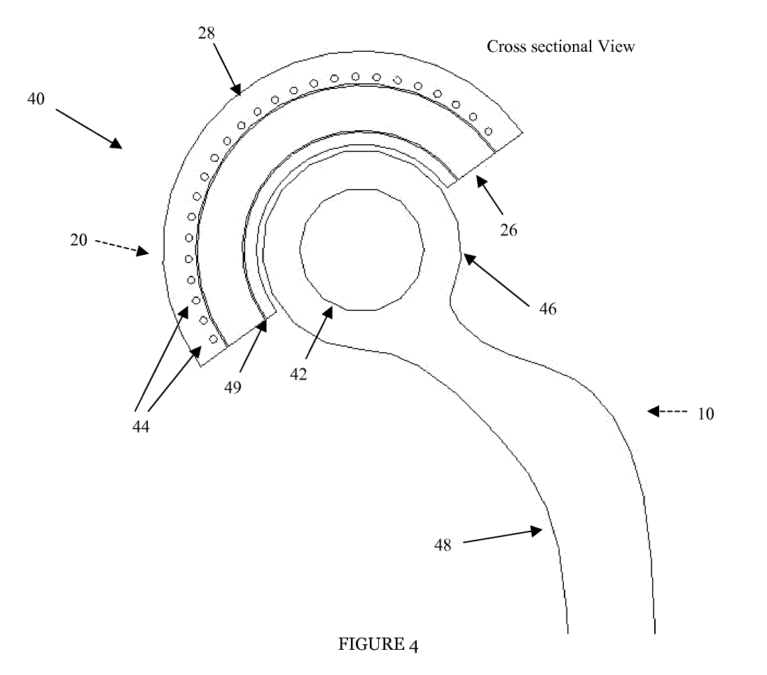

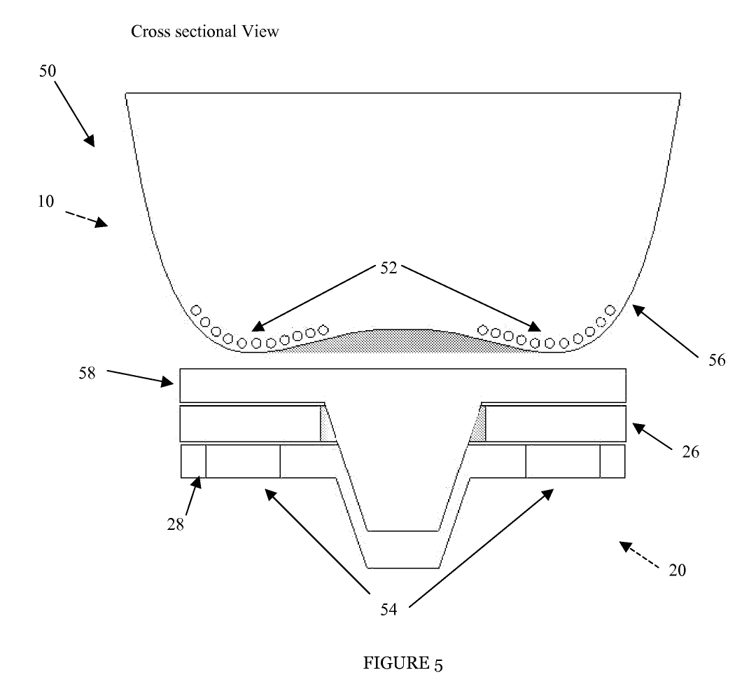

In the preferred embodiments, the first component 10 preferably moves relative to the second component 20, which remains relatively stationary to the body. The first component 10 may rotate, roll, or translate with respect to the second component 20. For example, in the first preferred embodiment 30 as shown in FIG. 3, the invention is applied to replace a shoulder joint and the first component 10 is the humeral component of the shoulder joint and the second component 20 is the glenoid component of the shoulder joint. In the second preferred embodiment 40, as shown in FIG. 4, the invention is applied to replace a hip joint and the first component 10 is the femoral head of the hip joint and the second component 20 is attached to the hip. However, based upon the joint geometry, joint location, force distribution, and other factors, any other combination of movement in the first and second components 10 and 20 may be used. For example, in the third preferred embodiment 50, as shown in FIG. 5, the invention may be applied to replace a knee joint and the first component 10 is attached to the femur and the second component 20 is attached to the tibia.

In the preferred embodiments, the first component joint interface 12 and the second component joint interface 22 are preferably of materials that, when in contact with each other, yield a very low coefficient of friction. The first component joint interface 12 is preferably of cobalt chrome. The first component 10 is also preferably composed entirely of the same material, for example, in a shoulder joint, the stem of the humeral head is preferably also of cobalt chrome. The second component joint interface 22 is preferably of Ultra High Molecular Weight Polyethylene (UHMWPE), which has a very low coefficient of friction when in contact with cobalt chrome. However, any other combination of materials that provides a suitably low coefficient of friction when in contact may be used.

In the preferred embodiments, the first component magnet arrangement 14 preferably creates a first magnetic field 15 that is uniform relative to the second magnetic field 25 throughout the range of motion of the joint. For example, for the range of motion of the joint and without compression of the compressible volume 26, the distance and strength of the first magnetic field 15 as seen by the second magnetic field 25 is approximately unchanged. This is preferably achieved using a plurality of magnets within the first component magnet arrangement 14 placed along or close to the surface of the first joint interface 12, as shown in FIGS. 1, 3, and 5. The magnets of the first component magnet arrangement 14 are preferably embedded into the material of the first joint interface 12 to prevent movement of the magnets upon contact between the first and second magnetic fields 15 and 25 and to prevent direct contact between the potentially non-biocompatible materials of the magnets with the body. Alternatively, as shown in FIG. 4, depending on the type of movement and the expected force distribution, the first component magnet arrangement 14 may consist of a single magnet. The magnets of the first component arrangement 14 are preferably neodymium magnets. However, any other magnet arrangement or magnet type within the first component magnet arrangement 14 suitable to the application may be used.

The second component magnetic field 24 preferably creates a second magnetic field 25 that is strong and localized and provides a strong repulsive force once in contact and overlap with the first magnetic field 15. This is preferably achieved using a single strong magnet placed behind the compressible volume 26, as shown in FIGS. 1 and 3. Alternatively, as shown in FIGS. 4 and 5, depending on the expected movement and force distribution, the second component magnet arrangement 24 may consist of a plurality of magnets placed behind the compressible volume 26. The magnets of the second component arrangement 24 are preferably neodymium magnets. However, any other magnet arrangement or magnet type within the second magnet arrangement 24 suitable to the application may be used.

The second component 20 may also include a backing 28 that encases the second component magnet arrangement 24 to prevent movement of the magnets relative to the second component 20 and prevents non-biocompatible materials form the magnet to come into direct contact with the body. When the first and second magnetic fields 15 and 25 are brought together, because of the strong nature of the fields, the created forces are very strong and the magnets of the first and second component magnet arrangements 14 and 24 will experience a strong tendency to move, either to bring like poles together or to pull towards each other. Thus, it is preferred that the magnets of both the first and second component magnet arrangements 14 and 24 are securely held to prevent any undesired motion of individual magnets. The backing 28 is preferably of a material with a high modulus of elasticity to prevent elastic deformation while under the high stresses that may be experienced during joint use. The material also preferably has a high Young's Modulus to prevent plastic deformation due to the high stresses that may be experienced during joint use. The backing 28 is preferably of a titanium material. Titanium is also a highly biocompatible material, is relatively light, and is used often in existing artificial joints. The backing 28 may also function to help anchor the second component 20 to healthy bone for implantation of the joint.

The compressible volume 26 functions to control the distance between the first and second magnetic fields 15 and 25 based upon the application of a compressive force. As shown in FIGS. 1-5, the compressible volume 26 is coupled to the second component 20 and is placed in between the first component magnet arrangement 14 and the second component magnet arrangement 24 and behind the second joint interface 22. As a result, the compressible volume 26 functions like a switch to control the separation between the first and second magnetic fields 15 and 25. Because it is placed behind the second joint interface 22, interaction properties of the compressible volume 26 are relatively unimportant and the material for the compressible volume 26 can be selected from a wide range of materials for the appropriate compressive properties. The compressible volume 26 preferably compresses to an amount that allows enough contact and overlap between the first and second magnetic fields 15 and 25 to create a repulsive force adequate to significantly reduce the overall normal force between the first and second components 10 and 20 and thus significantly decrease the amount of wear on the first and second joint interfaces 12 and 22. Depending on the application, the adequate force to accomplish the desired wear reduction may vary. The compressible volume 26 is preferably composed of an elastomer with a modulus of elasticity that allows adequate elastic compression of the compressible volume 26 when compressed with expected forces during joint use to provide the desired repulsive force for the application. When the forces are no longer applied, the elastomer preferably expands to the original volume. Alternatively, the compressible volume 26 may be of any other material type.

The following descriptions of the preferred embodiments include all of the features and functions as described above. Further embodiments may include use of the joint in mechanical bearings, linkages, braces, machinery, and any other suitable application where it may be beneficial to decrease or regulate the overall normal force within a joint.

### 1. First Preferred Embodiment

As shown in FIG. 4, the first preferred embodiment 30 of the invention is applied to a shoulder joint. In the first preferred embodiment, the first component 10 includes a humeral head element 36 as the first joint interface 12 and a stem element 38; both preferably made of cobalt chrome, but may alternatively be made of any other suitable material. The second component 20 includes a socket 39 as the second joint interface 22 and is preferably made of polyethylene, but may alternatively be made of any other suitable material. The humeral head 36 rotates, rolls, and translates relative to the socket 39. Because of this, first component magnet arrangement 12 preferably consists of a plurality of magnets 32 located beneath the convex surface of the humeral head 36 that provide a uniform first magnetic field 15 relative to the second magnetic field 25. The humeral head 38 preferably includes crevices, each to hold one magnet 32, preventing movement of the magnet relative to the first component 10 and to prevent contact of the magnet 32 with the body. The second component magnet arrangement 22 preferably consists of a single strong magnet 34 that provides a strong localized magnetic field 25 that creates a strong repulsive force when in significant overlap with the first magnetic field 15. The magnets 32 are preferably each cylindrical neodymium magnets of diameter ⅛ of an inch and thickness 1/16 of an inch that provide 0.92 lb of repulsive force when arranged with like magnetic poles in close proximity, such as K&J Magnets Inc. N42 D21 magnets. The strong magnet 34 is preferably a cylindrical neodymium magnet of diameter 1 inch and thickness ⅛ of an inch that provide 81.5 lbs of repulsive force when arranged with like magnetic poles in close proximity, such as K&J Magnets Inc N50 DX02 magnets. However, any other suitable arrangement, type, force attributes, and size of the magnets within the first and second component magnet arrangements 12 and 22 may be used.

The compressible volume 26 of the first preferred embodiment 20 is preferably an elastomer. The expected force between the first and second components 10 and 20 during joint use is approximately of the range 10N-400N. Thus, at the maximum expected force of 400N, the elastomer preferably compresses enough to allow for enough overlap of the first and second magnetic fields 15 and 25 to create the maximum repulsive force. With an assumption of a cylindrical compressible volume 26 with diameter 0.0254 meters with a thickness of 0.01 meters (uncompressed) and a desired compression distance of 0.008 meters (based upon the reach of the first and second magnetic fields 15 and 25), the desired modulus of elasticity is approximately 0.986 MPa. The compressible volume 26 of the first preferred embodiment is preferably an a Dynaflex® Polymer with a modulus of elasticity of 0.965 MPa. However, any other suitable material and arrangement for the compressible volume 26 may be used.

### 2. Second Preferred Embodiment

As shown in FIG. 5, the second preferred embodiment 40 of the invention is applied to a hip joint. In the second preferred embodiment, the first component 10 includes a femoral head 46 as the first joint interface 12 and a femoral stem 48, both preferably made of cobalt chrome, but may alternatively be made of any other suitable material. The second component 20 includes a socket 49 as the second joint interface 22, preferably made of polyethylene but may alternatively be made of any other suitable material, and an acetabular shell as the backing 28. The femoral head 46 mostly rotates relative to the socket 49. Because of this, the first component magnet arrangement 12 preferably consists of a single high gradient magnet 42 that is positioned within and concentric with femoral head 46. The high gradient magnet 42 functions to provide a significant increase in repulsive force with a small increase in overlap of the first and second magnetic fields 15 and 25. The high gradient magnet 42 may be produced by including a high concentration of neodymium at the center of the magnet 42 and a lower concentration of neodymium closer to the surface of the magnet 42, but may alternatively be produced using any other suitable method. The first component magnet arrangement 12 may alternatively be a matrix of a plurality of magnets arranged under the surface of the femoral head 46. The second component magnet arrangement 22 preferably consists of a plurality of magnets 44 placed behind the compressible volume 26 and concentric to the high gradient magnet 42 to provide a uniform relative second magnetic field 25. The plurality of magnets 44 are preferably embedded into the acetabular shell and held stationary relative to the second component 20, preventing direct contact with the body. However, any other suitable arrangement, type, force attributes, and size of the magnets within the first and second component magnet arrangements 12 and 22 may be used.

The compressible volume 26 of the second preferred embodiment 40 is preferably an elastomer placed concentric with the magnets 44 and the high gradient magnet 42, the plurality of magnets 44, and the femoral head 46 to provide equal compressive properties throughout the range of motion of the first component 10. However, any suitable material or arrangement of the compressible volume 26 may be used.

### 2. Third Preferred Embodiment

As shown in FIG. 6, the third preferred embodiment 50 of the invention is applied to a knee joint. In the third preferred embodiment, the first component 10 includes a femur cap 56, preferably made of cobalt chrome, as the first joint interface 12 and the second component 20 includes a tibia cap 58, preferably made of polyethylene, that includes a stem as the second joint interface 22. The second component 20 also includes a titanium disk with a stem that serves as the backing 28. The stem of the tibia cap 58 is inserted into the stem of the backing 28 and secured to the backing 28. The femur cap 56 rotates, rolls, and translates with respect to the tibia cap 58. Because of this the first component magnet arrangement 12 preferably consists of a plurality of magnets 52 located beneath the contact surfaces of the femur cap 56 that provide a uniform first magnetic field 15 relative to the second magnetic field 25. The femur cap 56 preferably includes crevices, each to hold one magnet 52, preventing movement of the magnet relative to the first component 10 and to prevent contact of the magnet 52 with the body. The second component magnet arrangement 22 preferably consists of a ring of magnets 54 embedded along the outer circumference of the titanium disk underneath the contact surface areas of the femur cap 56, providing an uniform magnetic field relative to the first magnetic field 15. However, any other suitable arrangement, type, force attributes, and size of the magnets within the first and second component magnet arrangements 12 and 22 may be used.

The compressible volume 26 of the third preferred embodiment 50 is preferably an elastomer. The elastomer is preferably a ring that surrounds the stem of the tibia cap 56, is placed in between the contact surface areas of the first and second components 10 and 20, and is supported by the stem of the titanium disk of the backing 28 to prevent shifting of the elastomer. Due to the geometry of the knee joint, stems and anchors are preferably used to anchor the components of the joint to each other. However, any other suitable material or arrangement of the compressible volume 26 may be used.

As a person skilled in the art of will recognize from the previous detailed description and from the figures and claims, modifications and changes can be made to the preferred embodiments of the invention without departing from the scope of this invention defined in the following claims.

We claim:

1. An artificial joint comprising:
a first component that includes a first component joint interface and a first component magnet arrangement that creates a first magnetic field;
a second component that includes a second component joint interface and a second component magnet arrangement that creates a second magnetic field;
a compressible volume located between the first component magnet arrangement and the second component magnet arrangement and adapted to control the overlap of the first and second magnetic fields;
wherein the first component joint interface, the compressible volume, and the second component joint interface cooperate to transfer the following compression loads:
a relatively low compression force—wherein the compressible volume reaches a low compression state, and wherein the first magnetic field and the second magnetic field create a low repulsion force that acts upon the relatively low compression force, and
a relatively high compression force—wherein the compressible volume reaches a high compression state that is more compressed than the low compression state, and wherein the first magnetic field and the second magnetic field create a high repulsion force that is greater than the low repulsion force and that acts upon the relatively high compression force.

2. The artificial joint of claim 1 wherein the first component joint interface is composed of cobalt chrome.

3. The artificial joint of claim 1 wherein second component joint interface is composed of Ultra High Molecular Weight Polyethylene (UHMWPE).

4. The artificial joint of claim 1 wherein the second component further includes a backing element that encases and secures the second magnet arrangement within the second component.

5. The artificial joint of claim 4 wherein the backing element is composed of titanium.

6. The artificial joint of claim 1 wherein the high repulsive force decreases the friction force between the first and second components, thus decreasing wear in the first and second joint interfaces.

7. The artificial joint of claim 6 wherein the first component moves relative to the second component during use of the artificial joint with motions selected from the group consisting of: rotation, rolling, translation.

8. The artificial joint of claim 7 wherein the first component magnet arrangement includes a plurality of magnets in an arrangement that provides a generally uniform first magnetic field relative to the second magnetic field throughout the range of motion of the first component.

9. The artificial joint of claim 8 wherein the magnets of the first component magnet arrangement are neodymium magnets.

10. The artificial joint of claim 9 wherein the magnets of the first component magnet arrangement are each cylinders of diameter ⅛ of an inch and thickness 1/16 of an inch.

11. The artificial joint of claim 10 wherein the magnets of the first component magnet arrangement each generate a repulsive force of approximately 1 lbs when arranged with like magnetic poles in close proximity.

12. The artificial joint of claim 8 wherein the plurality of magnets in the first component magnet arrangement are embedded into the first component.

13. The artificial joint of claim 7 wherein the second component magnet arrangement includes a single magnet.

14. The artificial joint of claim 13 wherein the single magnet of the second component magnet arrangement is a neodymium magnet.

15. The artificial joint of claim 14 wherein the single magnet of the second component magnet arrangement is a cylinder of diameter 1 inch and thickness ⅛ of an inch.

16. The artificial joint of claim 15 wherein the magnet of the second component magnet arrangement generates a repulsive force of approximately 80 lbs when arranged with like magnetic poles in close proximity.

17. The artificial joint of claim 14 wherein the magnet of the second component magnet arrangement is a spherical magnet.

18. The artificial joint of claim 14 wherein the magnet of the second component includes a first concentration of neodymium at a first position and a second concentration of neodymium at a second position, wherein the first position is interior to the second position and wherein the first concentration is greater than the second concentration.

19. The artificial joint of claim 7 wherein the second component magnet arrangement includes a plurality of magnets that provide a generally uniform strong second magnetic field relative to the first magnetic field throughout the range of motion of the first component.

20. The artificial joint of claim 6 wherein the first component joint interface, the compressible volume, and the second component joint interface cooperate to also transfer the following compression load: a relatively moderate compression force—wherein the compressible volume reaches a moderate compression state that is more compressed than the low compression state and less compressed than the high compression state, and wherein the first magnetic field and the second magnetic field create a moderate repulsion force that is greater than the low repulsion force and less than the high repulsion force.

21. The artificial joint of claim 6 wherein the compressible volume includes a volume of elastomer with a modulus of elasticity that allows elastic compression at a first and second amount of load resulting from joint use.

22. The artificial joint of claim 21 wherein the elastomer has a modulus of elasticity of approximately 0.986 MPa and a thickness of approximately 0.01 meter.

23. The artificial joint of claim 21 wherein the elastomer is a Dynaflex Polymer with a modulus of elasticity of approximately 0.965 MPa.

24. A method of managing forces between a first component and a second component of a joint comprising the steps of:
creating a first magnetic field from the first component;
creating a second magnetic field from the second component;
providing a compressible volume between the first and second magnetic fields;
upon the application of a relatively low compression force on the joint:
compressing the compressible volume to a low compression state, and creating a low repulsion force that acts upon the relatively low compression force, and
upon the application of a relatively high compression force on the joint:
compressing the compressible volume to a high compression state, and creating a high repulsion force that is more compressed than the low compression state, and creating a high repulsion force that is greater than the low repulsion force and that acts upon the relatively high compression force.

25. The method of claim 23 wherein the high repulsion force decreases the friction force between the first and second joint components and decreases wear in the first and second components.

26. The method of claim 23 wherein the first joint component moves relative to the second joint component during use and wherein the first magnetic field is generally uniform relative to the second magnetic field throughout the range of motion of the first joint component during use.

27. The method of claim 26 wherein the motion of the first joint component relative to the second joint component is of the type selected from the group consisting of: rotation, rolling, and translation.
